(19) United States
(12) Patent Application Publication
(10) Pub. No.: US 2009/0137034 A1
(43) Pub. Date: May 28, 2009

(54) METHODS FOR PERFUSION AND PLATING OF PRIMARY HEPATOCYTES AND A MEDIUM THEREFORE

(75) Inventors: John K. Leach, Roxbury Crossing, MA (US); Steven R. Tannenbaum, Framingham, MA (US)

Correspondence Address: RONALD I. EISENSTEIN;NIXON PEABODY LLP, 100 SUMMER STREET, BOSTON, MA, 02110, US

(73) Assignee: MASSACHUSETTS INSTITUTE OF TECHNOLOGY, Cambridge, MA (US)

(21) Appl. No.: 12/276,652

(22) Filed: Nov. 24, 2008

Related U.S. Application Data
(62) Division of application No. 10/521,920, filed on Jul. 11, 2005.
(60) Provisional application No. 60/399,493, filed on Jul. 30, 2002.

(51) Int. Cl. C12N 5/02

(52) U.S. Cl. 435/325

(57) ABSTRACT

The present invention provides methods for culturing primary hepatocytes with improved long term function and improved viability, by plating the hepatocytes in the presence of an anti-oxidant(s) as well as an agent(s) which is a functional inhibitor of enzymes that generate reactive oxygen and reactive nitrogen species. One preferred embodiment provides a combination of 2-oxo-thizolidine and tocopherol succinate. Another preferred embodiment provides a combination of N^{G}-methylarginine and mannitol.

### RELATED APPLICATIONS

The present application is a Divisional of U.S. Utility Application No. 10/521,920, currently pending which is a 371 National Stage of International Application No. PCT/US2003/023869 filed on Jul. 30, 2003, which designated the U.S., and which claims benefit under 35 U.S.C. § 119(e) of the U.S. provisional application Ser. No. 60/399,493, filed Jul. 30, 2002, the contents of which are incorporated herein by reference in their entirety.

### GOVERNMENT SUPPORT

This invention was supported in part by National Institutes of Health training grant and the government of the United States has certain rights thereto.

### FIELD OF THE INVENTION

The present application is directed to methods that enhance the long term function of primary hepatocytes.

### BACKGROUND OF THE INVENTION

Hepatocytes make up the bulk of the liver and are responsible for the liver's central role in metabolism and detoxification. Cultured hepatocytes are thus essential in the study of the pharmacology and toxicology of chemical entities in the liver. Primary cultures of adult normal human hepatocytes provide an in vitro model for investigating many of the aspects of liver physiology in mammals, preferably human, including for example the secretion of plasma proteins, the oxidative metabolism of drugs and xenobiotics and the activation of procarcinogens. In addition, such cultures provide a unique means to investigate the influence of physiopathological stimuli such as cytokines, growth factors and hepatotrophic viruses on liver specific functions under conditions which would not be possible in animals for ethical reasons.

Cultured hepatocytes have also been proposed in the construction of extracorporeal liver assist devices (LAD), which have been proposed as a treatment for patients suffering acute or fulminant liver failure. The LAD would function as a temporary support designed to provide hepatic function until liver transplantation or the regeneration of the patient's own liver. The LAD incorporates a bioreactor containing isolated hepatocytes that are expected to detoxify substances in the circulating plasma of patients in liver failure.

However, one of the challenges in using isolated hepatocytes for any of these applications is that many of these differentiated functions are transient, lasting only hours to a few days in culture. For example, primary hepatocytes grown using traditional cell culture techniques are typically viable for only three to seven days, and exhibit differentiated function for two to four days. Nishibe, Y, and Hirata, M. Induction of cytochrome P-450 isozymes in cultured monkey hepatocytes. Int J Biochem Cell Bio. 27:3:279-285. 1995. Jauregui, H O, Ng, S F, Gann, K L and Waxman, D J. Xenobiotic induction of P-450 PB-4 (IIB1) and P-450c (IA1) and associated monooxygenase activities in primary cultures of adult rat hepatocytes. Xeno, 21(9):1091-106. 1991. Niak, S, Trenkler, D, Santangini, H, Pan, J and Jauregui, H O. Isolation and culture of porcine hepatocyte for artificial liver support. Cell Trans 5:107-115, 1996. This limits their usefulness.

Hepatocyte cultures grown using current cell culture techniques lose specific structures including fenestrations, bile canniculi, and loss of binucleation. In addition, the cultured hepatocytes exhibit characteristics and structures not seen in the intact liver: they spread out and become elongated, and extend stress fibers. Soon after plating, hepatocytes decrease expression of some of the transcription factors that are responsible for inducing transcription of liver specific genes. Examples include, HNF-4, HNF-3, C-EBPα and C-EBPβ.

Another characteristic of fully differentiated hepatocytes is the secretion of albumin. Yet, in primary hepatocyte culture, albumin secretion is significantly decreased by day 3 and is nearly undetectable by ELISA methods by day 5.

Hepatocytes cultured in currently used medium lose the ability to metabolize xenobiotics soon after plating. Cytochrome P450 gene expression is greatly reduced by day 3 in culture. In addition to the expression of the drug metabolizing enzymes, the ability to induce expression of the drug metabolizing enzymes is also greatly reduced over time in culture.

The standard method for isolation of primary hepatocytes is using the two step collagen perfusion method. However, there is a measurable generation of reactive oxygen and reactive nitrogen species (ROS/RNS) during this procedure. ROS/RNS have pathological implications for the isolated hepatocytes and manifest themselves in the form of increased lipid peroxidation, protein modifications and damage of DNA.

Reactive oxygen species and reactive nitrogen species can play important roles in cells. For example, in response to various inflammatory stimuli, lung endothelial, alveolar, and airway epithelial cells, as well as activated alveolar macrophages, produce both nitric oxide (^{•}NO) and superoxide anion radicals (O₂^{•-}). ^{•}NO regulates pulmonary vascular and airway tone and plays an important role in lung host defense against various bacteria. However, ^{•}NO may be cytotoxic by inhibiting critical enzymes such as mitochondrial aconitase and ribonucleotide reductase, by S-nitrosolation of thiol groups, or by binding to their iron-sulfur centers. In addition, ^{•}NO reacts with O₂^{•-} at a near diffusion-limited rate to form the strong oxidant peroxynitrite (ONOO⁻), which can nitrate and oxidize key amino acids in various lung proteins such as surfactant protein A, and inhibit their functions.

Nitric oxide is formed by many types of cells in the body for the purpose of intercellular communication (brain, cardiovascular system) or as part of the immune or inflammatory response system (macrophages, endothelial cells). The chemistry of NO in oxygenated biological systems is very complex, both in number of chemical species and in a number of parallel and consecutive reactions.

DNA damage can occur from N₂O₃ when O₂ is also present. In this case, peroxynitrite forms rapidly and then, following protonation, undergoes homolytic scission to hydroxyl radical and NO₂.

Hepatocytes can be valuable systems for quantifying the effects of exposure to carcinogens. Some environmental chemicals pose risks to human health. The accurate assessment of these risks requires quantitative data on human exposure. Such data are currently estimated from measurements of the chemicals in the air, water, or food. Direct measurements in human blood, urine, or tissues have generally not been attempted, since the compounds involved are usually short-lived and present in low levels. Hepatocytes are useful for quantitation of toxicologically significant compounds by monitoring their reaction products to human proteins, including carcinogens, mutagens, and other reactive chemicals. Hepatocytes are also useful for more accurate assessments of human risk, and more precise epidemiological investigations of the links, for example, between exposure to carcinogens and human cancer.

Cultures of hepatocyte that remain either viable or functional for extended periods of time would be useful to liver biologists, toxicologists, and pharmaceutical researchers. Because of the short half life of the cultured hepatocyte, many phenomena cannot be studied properly. However, animals, including humans, encounter a myriad of toxicants in their environment. The first and most rigorous defense to such toxicants is the liver, which metabolizes over 80% of all agents that we intake.

The induction of cytochrome P450 enzymes, which function to clear toxic substances, is a major factor in whether or not a candidate compound goes forward in the drug discovery process. More precise predictions of P450 induction would represent a step forward in pharmaceutical production schemes. In the area of liver toxicology there can be a significant lag between exposure and manifestation of toxicity, for example, aflatoxin, a toxic metabolite formed by mold takes many days to have its effects on humans, to study these long range effects a hepatocyte that metabolized xenobiotics for extended amounts of time would be useful. Companies interested in determining the toxicity of their candidate compounds are obliged to test them for liver toxicity. Using isolated hepatocytes provides only an indication of short term problems. For example, if toxicity occurs after an exposure greater than 3 days, current hepatocyte culture would not detect the toxicity.

It would be desirable to have an improved method for maintaining hepatocytes in culture for extended periods of time for pharmaceuticals testing. Any drug targeted to treat the liver would be better tested in such an improved hepatocyte cell culture model. Potency and efficacy could better be modeled in a long lived hepatocyte model than in its short lived counterparts.

### SUMMARY OF THE INVENTION

The present invention provides methods that permit culturing primary hepatocytes for extended periods of time and maintain function and/or viability for greater than three days. The method comprises plating the hepatocytes in the presence of an anti-oxidant(s) and a second agent, wherein the second agent is (1) a functional inhibitor of an enzyme that generates reactive oxygen and reactive nitrogen species, or (2) directly inhibits the reactive species, or (3) increases intracellular glutathione.

In a preferred embodiment, the anti-oxidant is tocopherol succinate or a scavenger of the hydroxyl radical. Preferably, the hydroxyl radical scavenger is mannitol.

In another preferred embodiment, the second agent is a glutathione precursor or an inhibitor of nitric oxide. Preferably, the glutathione precursor is 2-oxo-thiazolidine. Preferably, the nitric oxide inhibitor is N^{G}-methylarginine.

A particularly preferred combination is 2-oxo-thiazolidine and tocopherol succinate.

Another particularly preferred combination is N^{G}-methylarginine and mannitol.

Another preferred embodiment provides a fusion molecule which fuses the anti-oxidant properties and the functional inhibitor of enzymes that generate reactive oxygen and reactive nitrogen species.

In one preferred embodiment, the method of the present invention is used during the isolation and culturing of intact liver. In another preferred embodiment, the method of the present invention is used for the isolation and culturing of primary hepatocytes. In another preferred embodiment, the method of the present invention is used for the isolation and culturing of liver slice cultures.

The present invention also provides methods for using the hepatocytes with improved long term function to screen compounds for long term toxicity during drug development. Such long-term function is greater than three days, more preferably greater than five days, still more preferably at least one week, even more preferably at least two weeks, and still more preferably at least one month.

The present invention also provides methods for using the hepatocytes with improved long term function to screen candidate drugs targeted to treat liver.

The present invention also provides methods for using the hepatocytes with improved long term function in a bioartificial liver device.

The present invention also provides methods for using the hepatocytes with improved long term function for the production of proteins, including those endogenously expressed in hepatocytes such as TNF-α and hepatocyte growth factor (HGF).

The present invention also provides methods for using the hepatocytes with improved long term function in the development of a cell culture model of hepatitis B and hepatitis C.

The present invention also provides methods for using the hepatocytes with improved long term function for cryopreservation of hepatocytes.

### BRIEF DESCRIPTION OF THE DRAWINGS

FIG. 1 depicts the CYP 1A1 induction mechanism.

FIG. 2 is a table describing the mechanism of redox control for various treatments.

FIG. 3 is a graph showing the fold induction of urea synthesis in hepatocytes treated with a variety of compounds, compared to an untreated control group.

FIG. 4 is a graph showing the fold induction of albumin secretion in hepatocytes treated with a variety of compounds, compared to an untreated control group.

FIG. 5 is a graph showing the fold induction of inducible CYP 1A1 activity in hepatocytes treated with a variety of compounds, compared to an untreated control group.

FIG. 6 is a graph showing basal CYP 1A1 activity in hepatocytes treated with a variety of compounds, compared to an untreated control group.

FIG. 7 shows a Western blot for CYP 1A1 in 3-MC treated hepatocytes.

FIGS. 8A and 8B show the results of treating rat primary hepatocytes with a variety of agents.

FIG. 9 shows that there is almost no EROD activity in the untreated group, but 3.5 fold in the treated group.

### DETAILED DESCRIPTION OF THE INVENTION

The present invention provides methods that permit primary hepatocytes by plating the hepatocytes in the presence of an anti-oxidant(s) as well as a second agent, wherein the second agent is (1) a functional inhibitor of an enzyme that generates reactive oxygen and reactive nitrogen species, or (2) an agent that directly inhibits the reactive species, or (3) an agent that increases intracellular glutathione. The hepatocytes of such cultures display function for periods of time greater than four days and/or viability that is greater than one week. One preferred embodiment provides a combination of 2-oxo-thiazolidine and tocopherol succinate. Another preferred embodiment provides a combination of N^{G}-methylarginine and mannitol.

The hepatocytes cultured by the present method display long-term function. Such a long-term function is defined as displaying at least one wild type differentiated function such as cytochrome P450 gene expression (sometimes EROD activity) xenobiotics metabolism, etc. and/or structures such as fenestrations, bile canniculi, binucleation, etc. for at least three days. Preferably, the hepatocytes demonstrate EROD activity. More preferably, multiple functions and structures. Preferably, the function(s) are displayed for at least five days, more preferably at least one week, still more preferably at least two weeks, even more preferably at least 16 days, yet more preferably at least three weeks and still more preferably at least one month. Even more preferably at least 50 days, yet more preferably, at least two months. Visibility is preferably maintained at least one week, more preferably at least two weeks, yet more preferably at least two weeks, and yet more preferably at least three weeks and still more preferably at least one month, yet more preferably at least two months.

The present invention also provides fusion molecules, in which the anti-oxidant and the second agent are present in the same molecule.

Oxidative stress is known to contribute or lead to a variety of diseases. For a review of diseases and disease conditions associated with oxidative stress, see Drugs of the Future, vol. 13 (10), p. 973 (1988) and Molecular and Cellular Biochemistry, vol. 84, p. 199 (1988).

Reactive oxygen species and reactive nitrogen species are well known in the art. For example, reactive oxygen species include but are not limited to superoxide (O₂), hydroxyl (OH); peroxyl (RO₂), alkoxyl (RO), and hydroperoxyl (HO₂) groups. Reactive nitrogen species include but are not limited to nitrogen oxide (NO) and nitrogen dioxide (NO₂).

Anti-oxidants function to scavenge free radicals and thereby prevent oxidative damage to cells. Treatments which function in redox control are well known in the art and include but are not limited to tocopherol succinate; mannitol, which scavenges hydroxy radicals; ebselen, which scavenges peroxynitrite and superoxide anion; NOHA, which generates nitric oxide; 1400W, which inhibits inducible nitric oxide synthase; n-acetyl cysteine, which increases intracellular glutathione levels; PDTC, which scavenges hydroxyl radical and superoxide anion; and glucosamine. Preferred anti-oxidants are tocopherol succinate and mannitol.

In addition to the anti-oxidant, the present invention provides for a second agent, which can be a functional inhibitor of an enzyme that generates reactive oxygen and reactive nitrogen species, or an agent that directly inhibits the reactive species, or an agent that increases intracellular glutathione.

Glutathione plays an important role in protecting cellular systems from oxidative damage. Glutathione is a detoxifying peptide that the body conjugates to xenobiotics (foreign chemicals or compounds) to render the xenobiotic more hydrophilic, thus promoting their excretion from the body. The synthesis of glutathione involves a two-step reaction, with the first being catalyzed by gamma-glutamylcysteine synthetase. Glutathione synthetase catalyzes the second reaction. Thus, as used herein, the phrases “increase in intracellular glutathione” or “elevation in intracellular glutathione” is intended to mean the glutathione tripeptide itself, as well as the enzymes responsible for its synthesis and conjugation to xenobiotics. Cysteine is an important amino acid and is the rate limiting substrate in the synthesis of glutathione. However, when administered directly, cysteine can be cytoxic. Cysteine prodrugs have been demonstrated to be effective in protecting cellular systems from various forms of stress. For these agents to be effective it is necessary for the prodrug to be cleaved either by enzymatic or non-enzymatic means. Once cysteine is released, it must be converted into glutathione to demonstrate a therapeutic effect.

One class of compounds which function as cysteine prodrugs is thiazolidine-4-carboxylates. (Cancer, Chemotherapy and Pharmacology, Vol. 28, p. 166 (1991) and Arch. Gerontology and Geriatrics, vol. 1, p. 299 (1982)). For example, the use of certain 2-substituted-thiazolidine-4-carboxylic acids as cysteine prodrugs in medicaments for delaying the onset of cataracts in mammals has been reported. However, none of the substituents defined by the reference imparts antioxidant or free radical scavenging properties to the reference 2-substituted-thiazolidine-4-carboxylic acid compounds. Similarly, U.S. Pat. No. 4,868,114 discloses a method of stimulating the biosynthesis of glutathione in mammalian cells by contacting the cells with an effective amount of certain L-cysteine prodrugs, and U.S. Pat. No. 4,952,596 discloses N-acyl derivatives of thiazolidine-4-carboxylic acid compounds which possess antipyretic, anti-inflammatory, mucolytic and analgesic activity in addition to activity in the treatment of ischemic pathologies and in pathologies caused by the overproduction of oxidant radicals. U.S. Pat. No. 5,846,988 discloses fusion compounds with an anti-oxidant phenolic portion fused to a thiazolidine-4-carboxylate portion for use as cytoprotective agents.

A preferred second agent is N^{G}-methylarginine, which inhibits nitric oxide.

Another preferred second agent is 2-oxo-thiazolidine, which is a cysteine-prodrug that functions as a glutathione precursor. Accordingly, the present invention also provides for preferred second agents which function to increase intracellular glutathione, including glutathione precursors.

The liver is remarkable in its ability to purge a wide spread of toxicants.

Critical to liver functions are the cytochrome P450 enzymes, which employ a chemistry to modify and catalyze clearance of toxic substances. The liver regulates these enzymes by inducing their expression after it encounters noxious agents. These enzymes are required for our survival; however, they also negatively mediate disease states in the context of certain combinations of medicines used to treat various ailments. This inadvertent induction of the cytochrome P450 enzymes can have pathological side effects. An example is the induction of the P450 enzymes when a person takes both alcohol and large doses of acetaminophen, which can result in acute liver failure.

Using our culture procedure, long term effects of drug candidates could be monitored. Currently, more than 140,000 drug candidates are brought into in vitro testing every year, but very few make it to the market. The failure of lead compounds is largely due to unpredicted toxicity and lack of efficacy over extended periods of time. A cell culture model that behaved more similarly to the in vivo condition would greatly enhance and streamline candidate testing of pipeline drugs.

The improved method for culturing hepatocytes is also useful in using such hepatocytes in a bioartificial liver device (BAL). Liver disease is a prominent health problem in the United States, one of the ten most common causes of death. The current standard of care treatment for patients suffering from acute or end-stage liver disease is to undergo liver transplantation. To exacerbate the situation, there are nearly four times as many patients that need donor livers than donors to donate them. This situation leads to more than half of all patients not receiving a needed liver. Due to expected increases in the incidence of hepatitis C infection, for which currently there is no vaccine or effective treatment, the need for a treatment for liver disease is estimated to triple by 2010. A BAL device can be used on patients whose liver function is compromised, much as dialysis is used for patients suffering from kidney failure. The market for an efficient bioartificial liver device is expected to increase greatly in the next decade. This is largely due to an increase in hepatitis infections world wide.

Hepatocytes can also be used for the production of proteins for therapies and for research, including the production of hepatocyte-related proteins such as TNFα and hepatocyte growth factor (HGF).

The hepatocytes can be used in a culture model of hepatitis B or hepatitis C, which does not currently exist. Indeed, such a primary hepatocyte culture should be able to assay for viral hepatitis infection and replication. Industrial and academic scientists have been struggling for many years to develop in vitro models of viral hepatitis. This in vitro model of hepatitis would facilitate high throughput screening of anti-viral agents as well as genetic engineering of the host cell to study host defense mechanisms.

These cultured hepatocytes can also be used in hepatocyte cryopreservation. Human hepatocyte availability is presently unpredictable. Thus, effective cryopreservation technique will aid researchers and clinicians alike. Currently, hepatocytes are shipped either plated on plastic or they are cryopreserved and sent frozen in a vial but have presently yielded mostly disappointing results. This present cultures of hepatocytes should avoid the problems of short-term function and viability.

### Assays for Hepatocyte Function

Any assay which characterizes the function of hepatocytes can be used to determine the long term function of the primary hepatocytes. Assays for hepatocyte functions are well known in the art and include but are not limited to assays for activity of hepatocyte enzymes, measuring proteins and compounds expressed by hepatocytes, and measuring expression levels of genes expressed in hepatocytes.

Assays for hepatocyte enzymes are known in the art. Hepatocyte enzymes include drug metabolizing enzymes such as CYP 1A1, aconitase, and other cytochromes. A preferred hepatocyte enzyme is CYP 1A1, including both basal activity as well as inducible activity. Assays for CYP 1A1 are described for example in Pierce et al., 1996. As used herein, EROD is ethoxyresorufin O deethylase activity, which is also known as p450 CYP 1A1, an important drug metabolizing enzyme, and is a marker of general drug metabolizing activity in primary hepatocytes. Cytochromes include cytochrome C (Clementi et al., 1996), and cytochrome P-450 isozymes such as P-450 PB-4 (IIB1) and cytochrome P-450 (IA1).

Proteins and compounds expressed by hepatocytes include but are not limited to urea, albumin secretion, and CYP 1A1. For example, the secretion of albumin is characteristic of fully differentiated hepatocytes. Albumin secretion can be measured using well known standard ELISA assays, for example as described in Dunn et al., 1991. Urea synthesis can be measured by detecting the conversion of urea to indophenol, as described in Fawcett et al., 1960. Other proteins expressed in hepatocytes include TNF-α and hepatocyte growth factor (HGF).

Genes which are expressed in hepatocytes include transcription factors that are responsible for inducing transcription of liver specific genes. Examples include, HNF-4, HNF-3, C-EBPα and C-EBPβ. The expression levels of these genes can be detected using assays well known in the art, including Northern blotting to measure mRNA levels, quantitative rt PCR, and Western blotting to measure protein levels.

### Hepatocyte Culture Systems

The present invention can use any hepatocyte culture system, which are well known in the art. Preferred hepatocyte culture systems include primary hepatocyte cultures, hepatocyte cell lines, liver slice cultures, and explanted cultures of whole livers.

Any source of hepatocytes can be used. Hepatocytes to be subjected to the primary culture may be any cells constituting the liver of a mammal, and such cells can be isolated from the liver of an animal in a method known in the art. The liver donor for obtaining hepatocytes is preferably a normal or transgenic animal donor of either the mammalian or rodent species, more preferably of equine, canine, porcine, ovine, or murine species.

For certain applications such as a bioartificial liver device, a porcine donor is presently preferable. Due to the ease of handling smaller animals and liver organs, pigs between about 5 kg to about 20 kg are used, preferably about 8 kg, but any size donor may be used as a source for liver organs. Other mammalian sources including humans can also be used.

In another embodiment of the invention, the hepatocytes are induced. Hepatocytes may be induced in vivo, before they are isolated from the animal's liver, or in vitro, after isolation of the hepatocytes.

Induction in vivo is preferably performed by administering at least one induction agent to an animal donor via direct injection to the bloodstream, intraperitoneally, or intramuscularly; however, induction agents may also be administered to a donor using other routes such as orally, transdermally, or by inhalation. One or more induction agents may be administered at one time in a single dose or over a period of time as separated doses of different induction agents. The donor may be dosed with a combination of two or more induction agents to upregulate certain desired detoxification enzymes to crate a hepatocyte culture having a customized enzyme activity profile. The dosing of the induction agent may be administered in a single day or over a period of time, such as over a number of hours or days, before isolating the hepatocytes from the donor liver. For example, some induction agents such as phenobarbital are relatively unstable molecules after injection to a donor and are, therefore, more effective if provided at multiple intervals prior to procuring the organ. The amount of the induction agent in the dose depends on (1) the induction agent or agents used, (2) the species, size, and sex of the donor, (3) the mode of administration of at least one induction agent, and (4) the frequency of dose administration. Typically, when the induction agent is administered over a series of doses, the dosage of induction agent may be less. One of skill in the art would be able to successfully determine how to manipulate these dosing parameters in order to obtain in vivo induced hepatocyte cultures for use in the methods of the present invention.

Any induction agent that is known in the art may be used. Induction agent means an agent that is capable of increasing or upregulating hepatocyte cell functions, particularly those enzymes involved with detoxification, particularly cytochrome P450 or the conjugative reactions involved in detoxification. It is also useful if the induction agent maintains or improves other hepatocyte cell functions including metabolic functions such as ammonia clearance and synthetic functions such as albumin and transferrin production.

Induction agents are selected from the group including but not limited to: beta-napthoflavone (BNF), phenobarbital, 3-methycholanthrene (3MC), ethanol, dexamethasone, arochlor 1254, 2,3,7,8-tetrachlorodibenzo-p-dioxin, phenothiazine, chlorpromazine, isosafole, gamma-chlordane, allylisopropylacetamide (AIA), trans-stilbene oxide, kepone, acetone, isoniazid, pyridine, pyrazole, 4-methylpyrazole, pregneolone 16-alpha-carbonitrile (PCN), troleandomycin (TAO), clotrimazole, clofirate, clobuzarit, di(2-ethylhexyl)phthalate (DEHP), or mono-(20ethyhexyl)phthalate (MEHP). Particularly preferred induction agents are 3-methylcholanthrene, beta-napthoflavone (BNF), and phenobarbital. 3-methylcholanthrene is the most preferred inducing agent.

One or more induction agents may be used in vivo to upregulate the enzymatic activity of the hepatocytes prior to isolation. A single induction agent may be administered to a donor one or more times prior to isolation. Induction agents may be combined, meaning as a mixture or a cocktail at the same time, or serially, meaning separately at different times, when administered to upregulate a profile of target enzymes. The amount of induction agent contained in the dose should be enough to induce the hepatocytes to increase their functional metabolic activity but not so much as to be lethal to the liver organ or the donor. The time that the induction agent is provided to a donor should be long enough to result in upregulation of enzymatic detoxification activity, preferably at least about 24 hours prior to isolation.

Dosages for these inducing agents and additional agents are described in U.S. Pat. No. 6,394,812.

If a recipient patient is in need of liver assist treatment for an indication where the expression of detoxification enzyme activity is low, a liver assist device may be prepared using a mixture of cell isolates having a profile of hepatocytes with a number of enzyme activities upregulated to achieve the greatest range of detoxification activity and provide a tailor-made culture for treatment of acute failure.

For isolation of primary hepatocytes, any method known in the art can be used. A preferred method is the two step collagenase perfusion isolation method.

For example after the induction stage, hepatocytes can be isolated using a modification of the Seglen hepatocyte isolation method, as described in Seglen, P O. Preparation of isolated rat liver cells. In Methods in Cell Biology (D M Prescott, ed.) vol. 13. Academic Press (New York, N.Y.), 1976, incorporated herein. The animal is anesthetized, opened, and the exposed liver is cannulated and perfused in situ with cold lactated Ringers solution before excision to rinse blood and excess induction agent from the liver tissue. The excised liver is transported to a biological safety cabinet where the remainder of the procedure may be performed under aseptic conditions. The extracellular matrix that provides the physical structure of the liver is then digested by quickly perusing the organ with warmed EDTA, preferably at 37° C., followed by perfusion of 1 mg/ml collagenase at 37° C. until digestion appears complete (mean digestion time is about 22 minutes). Further digestion is then stopped with the addition of cold Hank's Balanced Salt Solution (HBSS) supplemented with calf serum. Digestion of liver matrix releases cells and cell aggregates from the matrix structure to create a suspension of cells. Undigested tissue and gallbladder are excised and the remainder of the tissue is passed through 200 micron and 100 micron stainless steel sieves to release cells and cell aggregates. The cell suspension is washed twice by centrifugation and decanting of rinse media and the cell pellet resuspended in media preferably after the third rinse. At this point, cells may be cultured in culture medium or cryopreserved in a cryopreservation medium for long-term storage for future use.

The cells are cultured as a cell suspension or plated on a surface suitable for animal cell or tissue culture, such as a culture dish, flask, or roller-bottle, which allows for hepatocyte culture and maintenance. The cells may be incorporated in a bioreactor, either in suspension or plated on a culture substrate such as a culture bead or fiber, or on a flat surface or membrane. Suitable cell growth substrates on which the cells can be grown can be any biologically compatible material to which the cells can adhere and provide an anchoring means for the cell-matrix construct to form. Materials such as glass; stainless steel; polymers, including polycarbonate, polystyrene, polyvinyl chloride, polyvinylidene, polydimethylsiloxane, fluoropolymers, and fluorinated ethylene propylene; and silicon substrates, including fused silica, polysilicon, or silicon crystals may be used as a cell growth surfaces. To enhance cell attachment or function, or both, the cell growth surface material may be chemically treated or modified, electrostatically charged, or coated with biologicals such as with extracellular matrix components or peptides. In one embodiment, the hepatocytes are cultured either within or on the surface of extracellular matrix disposed on the culture surface such as collagen in the form of a coating or a gel. In another embodiment, the hepatocytes are cultured on either a liquid-permeable membrane or a gas-permeable membrane. Other cells present in liver may also be included with the induced hepatocytes such as endothelial cells; Kupfer cells, a specialized macrophage-like cell; and, fibroblasts. A co-culture of hepatocytes with one or more of these or other types of cells may be desirable to optimize hepatocyte functioning.

In one embodiment, the in vivo induced hepatocytes can be seeded in a bioreactor that is used as, or is incorporated into a LAD. Some LAD designs are based on a hollow fiber cartridge design where the hepatocytes are seeded either in the lumen of the hollow fibers or on the outside of the hollow fibers. The hollow fiber serves as a culture substrate that allows for liquid or gas transport across the hollow fiber. Other LAD designs incorporate a flat planar culture substrate. Hepatocyte culture between two collagen gel layers is described in U.S. Pat. Nos. 5,602,026, and 5,942,436 to Dunn, et al. Another design using a planar culture substrate is disclosed in U.S. Pat. No. 5,658,797 and in International PCT Publication No. WO 96/34087 to Bader, et al. Some flat planar substrates may be micropatterned so that two or more cell types may be cultured together, as a co-culture, in discrete regions on a substrate such as those described by Bhatia, et al. The disclosures of these aforementioned patents that disclose culture substrates and methods and their use as a bioreactor device to treat patients in need of liver assist are incorporated herein by reference.

A preferred bioreactor design for the culture of hepatocytes incorporates a gas-permeable, liquid impermeable membrane that defines two regions of a bioreactor chamber. Hepatocytes are seeded on the surface of the membrane cultured in a liquid medium while engaging in oxygenation and other gas transfer not only in the culture medium but also across the membrane. In alternative embodiments, the membrane is treated to improve cell adhesion such as by modifying the electrical charge of the membrane, as by corona discharge, or by treating or coating the membrane with extracellular matrix components, peptides, cell-adhesion molecules or other chemicals. A preferred coating for the membrane is collagen.

When cultured, the cells are preferably contacted with a cell culture medium for a time to maintain their metabolic activity and optimal hepatocyte function. Albeit in varying concentrations, cell culture media provide a basic nutrient source for cells in the form of glucose, amino acids, vitamins, and inorganic ions, together with other basic media components. Culture media generally comprises a nutrient base further supplemented with one or more additional components such as amino acids, growth factors, hormones, anti-bacterial agents and anti-fungal agents. One preferred medium for use in the method after hepatocyte isolation comprises: Williams' E medium, newborn calf serum (NBCS), glucose, insulin, glucagon, hydrocortisone, HEPES, epidermal growth factor (EGF), and glutamine. In a more preferred embodiment, the culture medium which is supplemented with the present anti-oxidants and second agent comprises: Williams' E media supplemented with up to 1% newborn calf serum (NBCS), 4.5 g/l glucose, 0.5 U/ml insulin, 7 ng/ml glucagon, 7.5 μg/ml hydrocortisone, 10 mM HEPES, 20 ng/ml EGF, and 200 mM glutamine. Other concentrations for the aforementioned medium components or their functional equivalents may be determined for use by one of skill in the art of hepatocyte culture.

Another preferred medium for culturing hepatocytes makes use of a medium to which pleiotrophin (PTN), fetal bovine serum (FBS) and nicotinamide or an analogue thereof have been added. More preferably, the medium further contains an ascorbic acid or an analogue thereof (e.g. L-ascorbic acid phosphate). To this medium the antioxidants and second agents described above are also added to the basic medium.

For example, in one embodiment of the culture method described in Japanese Patent Application No. 133985/1996), hepatocytes are cultured in a mixed medium consisting of a medium containing FBS and nicotinamide or an analogue thereof as colony-forming ingredients for hepatocytes and a conditioned medium (CM) of 3T3 cells as a growth promoting factor for hepatocytes, where PTN is added in place of a CM of 3T3 cells to the medium of said prior application.

The compound PTN is one of heparin-binding proteins, and its action as growth factor and trophic factor for nerve cells is known. Some researchers have reported its action of promoting division of somatic cells such as fibroblasts, endothelial cells, epithelial cells etc., but there are also reports negating such action, so the effect of PTN on somatic cells is not established (The Journal of Biological Chemistry, Vol. 267, No. 36, pp. 25889-25897, 1992).

The compound PTN is commercially available as recombinant human PTN (R&D Systems Inc.), and this commercial product can also be used in the method of the present invention. Using the isolation and purification procedures described in U.S. Pat. No. 6,136,600, PTN can also be obtained from a CM of 3T3 cells prepared in the same manner as in Japanese Patent Application No. 133985/1996 or from a culture supernatant of other animal cells. Alternatively, PTN derived from 3T3 cells may be obtained by using a part of a known amino acid sequence of PTN as a probe to isolate a coding sequence of PTN from an existing cDNA library and then expressing this coding sequence in a suitable host-vector system.

The medium to which PTN, FBS, ascorbic acid or an analogue thereof and nicotinamide or an analogue thereof are added is specifically DMEM medium containing epidermal growth factor and DMSO. The epidermal growth factor (EGF) and DMSO are not essential for colony formation but are preferably added to the medium by virtue of their action of promoting colony formation. A fraction obtained by low-speed centrifugation contains endothelial cells, Kupffer's stellate cells, stellate cells, bile-duct epithelial cells in addition to hepatocytes and is considered to provide hepatocytes with a specific environment, and said nicotinamide or an analogue thereof, ascorbic acid or an analogue thereof and DMSO inhibit the growth of these non-parenchymal cells, thus making it possible to selectively culturing and proliferating parenchymal hepatocytes. The amounts of these ingredients added to the medium can be, for example, about 0.1 ng/ml to 10 μg/ml for PTN, 5 to 30% for FBS, 0.1 to 1.0 mM for ascorbic acid or an analogue thereof, 1 to 100 ng/ml for EGF, 1 to 20 mM for nicotinamide or an analogue thereof, and 0.1 to 2% for DMSO. Culture is conducted in a 5% CO.sub.2 atmosphere at a temperature of about 37° C.

In an alternate preferred embodiment, hepatocytes are cryopreserved for storage after isolation until needed for incorporation in a bioreactor. Cryopreservation of cell suspensions, cell monolayers, and engineered tissue constructs are known in the art of cryopreservation. Cryopreservation is useful for long term storage, banking, and shipping. When needed, the cultures are removed from frozen storage, thawed, rinsed of cryopreservative, and ready for use.

After either isolation or removal from cryopreservation storage, in one preferred embodiment the in vivo induced hepatocytes are preferably incorporated and cultured in a bioreactor. Hepatocytes from a single isolation induced with either a single or multiple doses of the same induction agent, or a number of induction agents, may be used. In one alternative embodiment, hepatocytes isolated from a non-induced donor are cultured in a bioreactor with hepatocytes isolated from an in vivo induced donor. In an another alternative embodiment, hepatocytes from two or more donor isolations induced by the same induction agent or at least two different induction agents are combined together in the same bioreactor. If the bioreactor has multiple culture chambers or regions, hepatocytes from different donors that have been pre-treated with different induction agents may be segregated but used together for the overall functioning of the bioreactor. Combining in vivo induced hepatocyte cultures that have different enzyme activity profiles in a bioreactor used as a LAD will benefit a patient treated with the cultures in the bioreactor. In one embodiment, the bioreactor may contain several isolations of different in vivo induced hepatocyte cultures to provide the patient with a full profile of upregulated enzymes to achieve the greatest range of detoxification activity. An alternative embodiment is one where the patient may be treated with a bioreactor seeded with one or more isolations of in vivo induced hepatocytes with certain selected enzymatic activities that augment or replace certain levels of enzymatic activity where the patient's liver expresses low levels of a certain detoxification enzyme.

The bioreactor may be used to culture the cells to produce cell products or to functionally act on substances, such as toxins normally metabolized by liver. The bioreactor may serve as, or be an integral part of, a liver assist device to treat a patient in need of liver assist. Hepatocytes having upregulated enzymatic activity may be used in various types of bioreactors used as liver assist devices. Bioreactors suited for this purpose comprise suspension means, hollow fibers, radial flow surfaces and planar substrates as cell culture.

Hepatocytes that have been induced in vivo are useful to treat a patient in need of liver assist when cultured in a bioreactor that is used as, or is incorporated into, a liver assist device. Usually, hepatocyte perfusion medium and the patient's plasma or blood are circulated through the device in separate flow loops. The flow loops contact each other via a membrane for the exchange of gases, toxins, and albumin but also provide an immunological barrier between the hepatocytes and the patient.

### Applications

An improved hepatocyte would be useful to liver biologists, toxicologists, and pharmaceutical researchers. Because of the short half life of the cultured hepatocytes using standard methods, it has not been possible to study many phenomena properly.

Accordingly, the present invention provides methods for using the hepatocytes with improved long term function to screen compounds for long term toxicity during drug development. The induction of P450 enzymes is a major factor in whether or not a candidate compound goes forward in the drug discovery process. More precise predictions of P450 induction would represent a step forward in pharmaceutical production schemes. In the area of liver toxicology, there can be a significant lag between exposure and manifestation of toxicity. Standard methods of culturing primary hepatocytes only result in cells which remain functional for a few days; however, many agents do not exhibit their toxic effect immediately. For example, aflatoxin, a toxic metabolite formed by mold, takes many days to have its effects on humans. Thus, the hepatocytes of the present invention are useful to study these long range effects of metabolized xenobiotics for extended amounts of time.

Currently, more than 140,000 drug candidates are brought into in vitro testing every year, but very few make it to the market. The failure of lead compounds is largely due to unpredicted toxicity and lack of efficacy. A cell culture model that behaved more similarly to the in vivo condition would greatly enhance and streamline candidate testing of pipeline drugs. The hepatocytes of the present invention are useful in determining the toxicity of candidate compounds during drug development, which must be tested for liver toxicity. This can be accomplished on isolated hepatocytes, but if the toxicity occurs after a prolonged exposure, greater than 3 days, current methods of hepatocyte culture would not detect the toxicity. Using the hepatocytes of the present invention, long term effects of drug candidates can be monitored.

The present invention also provides methods for using the hepatocytes with improved long term function to screen candidate drugs targeted to treat liver. Drugs which are targeted to treat the liver can be better tested in the long term cultures of the present hepatocytes; the improved function of the hepatocytes affords improved testing of potency, efficacy, and toxicity.

The present invention also provides methods for using the hepatocytes with improved long term function in a bioartificial liver device. The need for an efficient bioartificial liver device is anticipated to increase greatly in the next decade, largely due to an increase in hepatitis infections world wide.

The present invention also provides methods for using the hepatocytes with improved long term function for the production of proteins, including those endogenously expressed in hepatocytes such as TNF-α and hepatocyte growth factor (HGF), for use in both therapeutic and research applications.

### EXAMPLE 1

### Modulation of Reactive Oxygen and Nitrogen Species in Freshly Isolated Hepatocytes, and Its Effect on Hepatocyte Phenotype and Function

To study the effect of different compounds on the function of hepatocyte function and phenotype, hepatocytes were isolated according to techniques well known in the art and cultured in a collagen sandwich configuration. Hepatocytes were induced with the addition of 2 nM 3-methylcholanthrene (3-MC). CYP 1A1 was measured using the EROD assay as described in Pearce et al., 1996. Albumin secretion was measured using ELISA, as described in Dunn et al., 1991. Western blot analysis was performed using techniques well known in the art, using an antibody to CYP1A1.

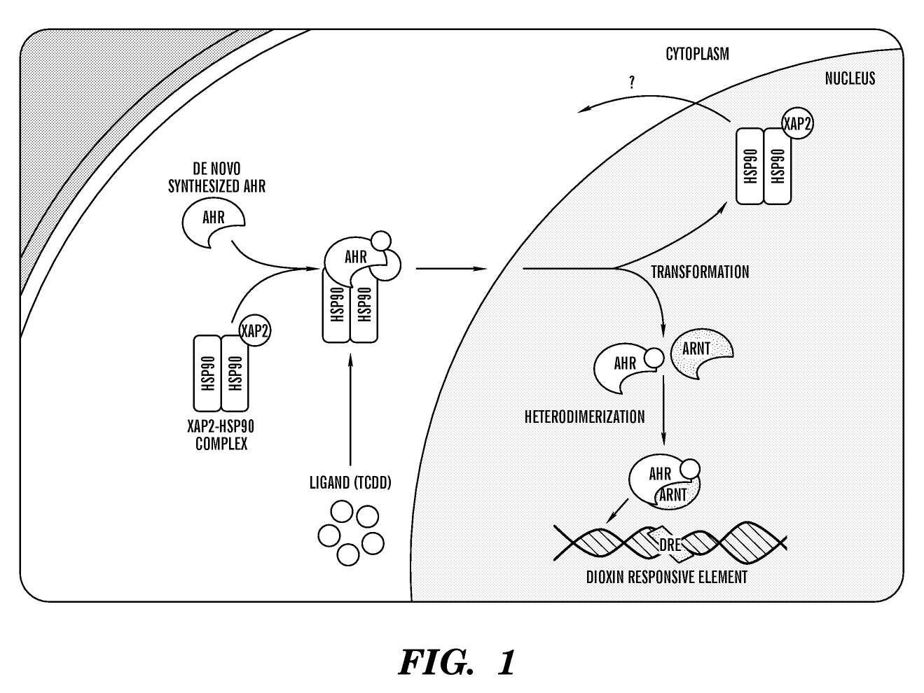

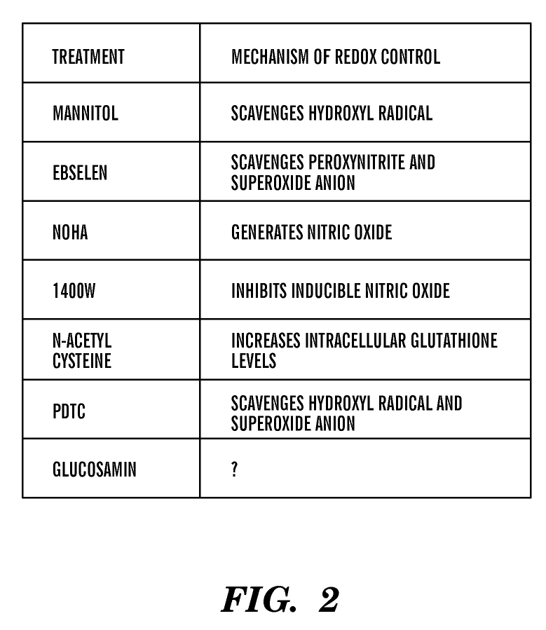

The CYP 1A1 induction mechanism is depicted in FIG. 1. FIG. 2 is a table describing the mechanism of redox control for various treatments.

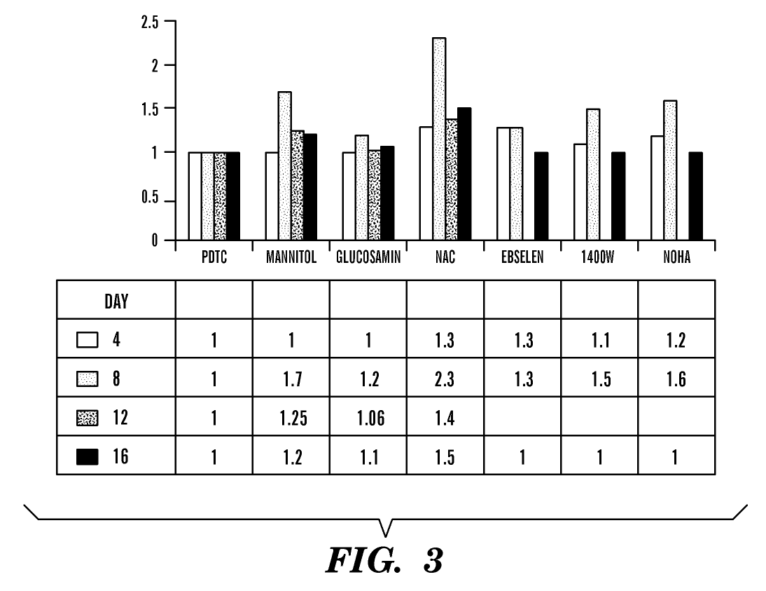

FIG. 3 is a graph showing the fold induction of urea synthesis in hepatocytes treated with a variety of compounds and cultured for 16 days, compared to an untreated control group.

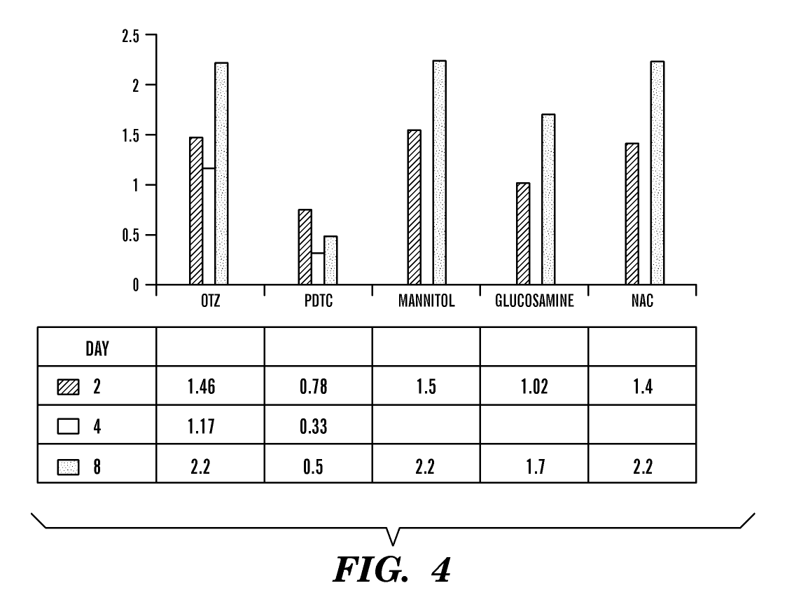

FIG. 4 is a graph showing the fold induction of albumin secretion in hepatocytes treated with a variety of compounds and cultured for 8 days, compared to an untreated control group.

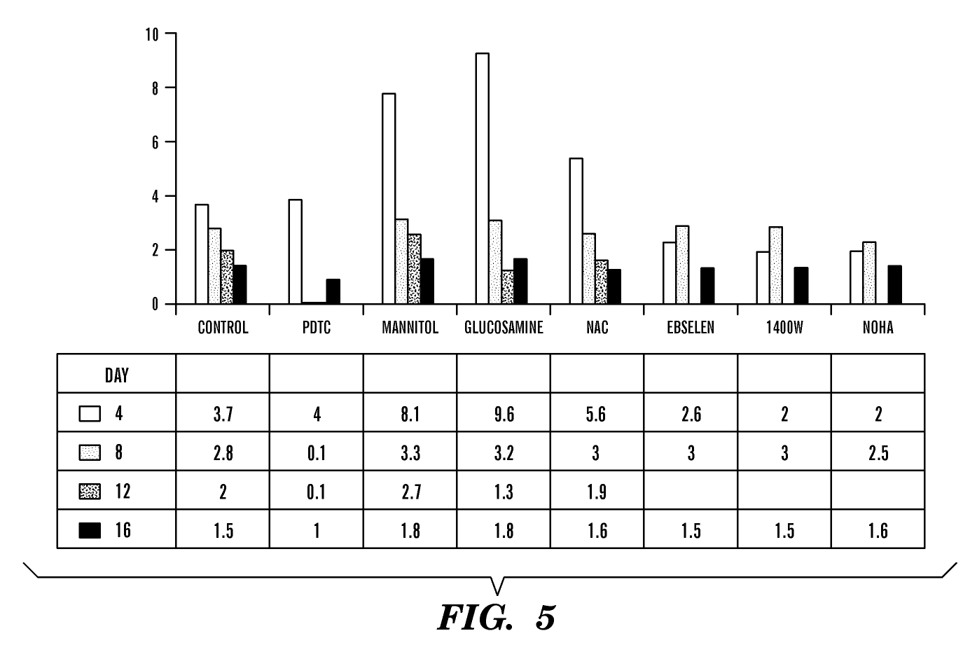

FIG. 5 is a graph showing the fold induction of inducible CYP 1A1 activity in hepatocytes treated with a variety of compounds and cultured for 16 days, compared to an untreated control group.

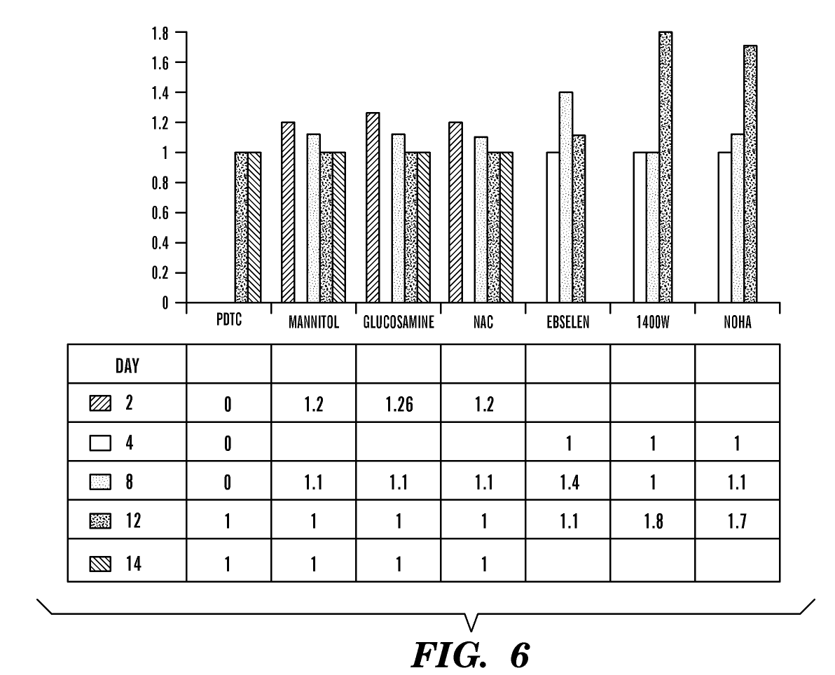

FIG. 6 is a graph showing basal CYP 1A1 activity in hepatocytes treated with a variety of compounds and cultured for 14 days, compared to an untreated control group.

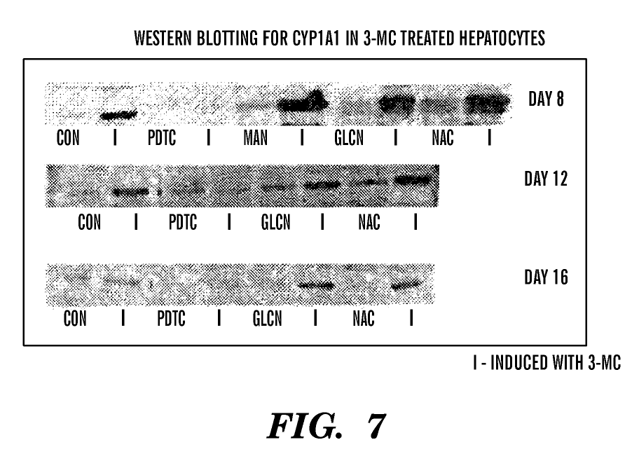

FIG. 7 shows a Western blot for CYP 1A1 in 3-MC treated hepatocytes, cultured for 8, 12 and 16 days.

These experiments indicate that antioxidants are effective at increasing CYP 1A1 inducibility in primary hepatocytes. NFkB nuclear translocation is critical to hepatocyte function. The benefit of nitric oxide (probed by either inhibiting nitric oxide or donating nitric oxide) was measurable but minimal. Amongst the class of anti-oxidants, scavenging the hydroxyl radical or increasing intracellular glutathione was most effective in maintaining P450 function. The mechanism of glucosamine protection of P450 function is under investigation, and may function as an antioxidant based on the observation that glucosamine inhibits NO synthesis in activated macrophages.

### EXAMPLE 2

### Rat Primary Hepatocytes

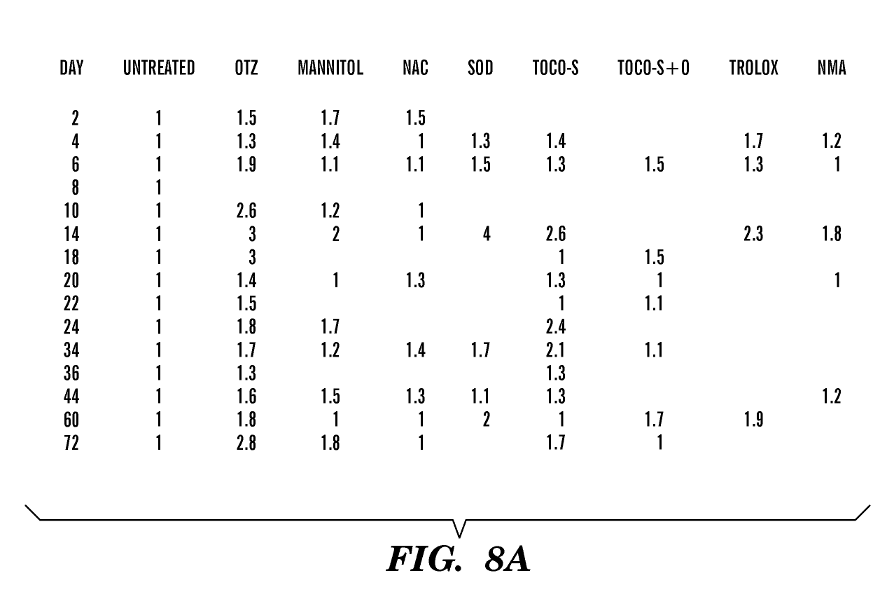

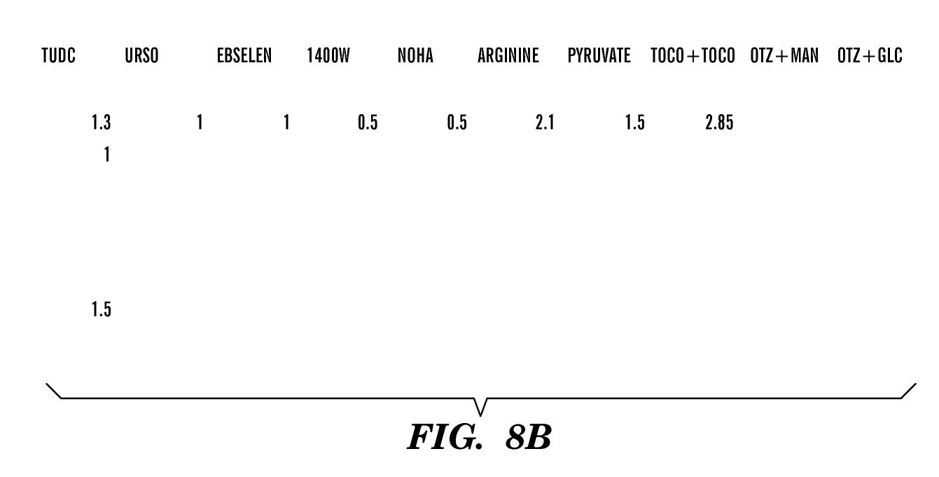

The function of rat primary hepatocytes prepared using the methods of the present invention were analyzed. The average fold increases in EROD activity in rat primary hepatocytes treated and untreated was determined, as shown in FIG. 8. The numbers are averages, with levels greater than 1.2 being significant. EROD is ethoxyresorufin O-deethylase activity, which is also known as p450 CYP 1A1, an important drug metabolizing enzyme, and is a marker of general drug metabolizing activity in primary hepatocytes.

### EXAMPLE 3

### Mouse Primary Hepatocytes

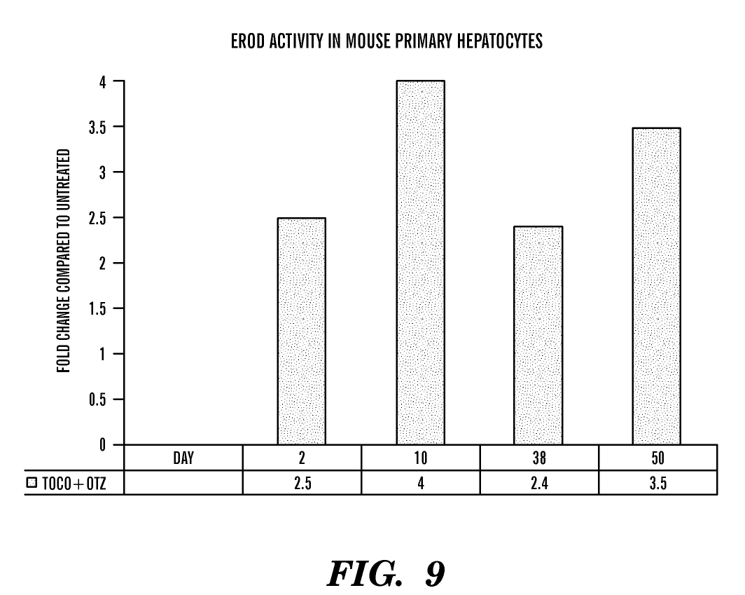

The function of mouse primary hepatocytes using the methods of the present invention was also examined. Using OTZ and tochpheral succinate using the present method, we were able to maintain a mammalian hepatocyte for greater than 50 days after isolation as well as maintaining EROD activity for such a period. See FIG. 9. There is almost no EROD activity in the untreated group, but 3.5 fold in the treated group. Experiments were done in quadruplicate, with the average shown.

### REFERENCES

1. Ziemann et al., Carcinogenesis, 20(3);407-14 (1999);
2. Adamson et al., Toxicol Appl. Pharmacol., 119(1):100-7 (1993);
3. Zhang et al., Chem. Biol. Interact., 138(3):267-84 (2001);
4. Andres et al., J. Hepatol., 33(4):570-9 (2000);
5. Tirmenstein, et al., Chem. Biol. Interact., 127(3):201-17 (2000);
6. Ray et al., Arch. Biochem. Biophys., 311(1):180-90 (1994);
7. Roberts et al., J. Med. Chem., 30(10):1891-6 (1987);
8. Larrauri, et al., Mol. Toxicol., 1(4):301-11 (1987-88);
9. Inoue et al., Ann. Surg., 218(3):350-62 (1993);
10. Kim et al., Hepatology, 32 (4 Pt 1):770-8 (2000);
11. Guillouzo, A., Environ. Health Perspect., 106(2):511-532 (1998);
12. Clementi et al., Proc. Natl. Acad. Sci., pp. 1559-1562 (1996);
13. Castro et al., J. Biol. Chem., 269:29405-29415 (1994);
14. Khastenko et al., Proc. Natl. Acad. Sci., 90:11147-11151 (1993);
15. Fawcett et al., J. Clin. Pathol., 13:156 (1960);
16. Pearce et al., Arch. Biochem. Biophys., 331:145-169 (1996);
17. Dunn et al., Biotechnol. Prog., 7(3):238-245 (1991).

All references described herein are incorporated herein by reference.

We claim:

1. A method for culturing primary hepatocytes comprising plating primary hepatocytes in the presence of an anti-oxidant(s) and a second agent, wherein said second agent is (1) a functional inhibitor of an enzyme that generates reactive oxygen and reactive nitrogen species, (2) an agent that directly inhibits the reactive oxygen or the reactive nitrogen species, or (3) an agent that increases intracellular glutathione and culturing said primary hepatocytes for at least five days, wherein said primary hepatocytes maintain function to metabolize xenobiotics or cytochrome P450 gene expression for at least five days, wherein the cultured primary hepatocytes are further exposed to a candidate compound for more than 3 days.

2. The method of claim 1, wherein the cultured primary hepatocytes are further exposed to a candidate compound for more than 5 days.

3. The method of claim 1, wherein the cultured primary hepatocytes are further exposed to a candidate compound for more than one week.

4. The method of claim 1, wherein the cultured primary hepatocytes are further exposed to a candidate compound for more than two weeks.

5. The method of claim 1, wherein the cultured primary hepatocytes are further exposed to a candidate compound for more than one month.

6. The method of claim 1, 2, 3, 4, or 5, wherein the candidate compound is a drug candidate.

7. The method of claim 1, wherein the cultured primary hepatocytes are further exposed to hepatitis after 5 days in culture.
